(19) United States
(12) Patent Application Publication
(10) Pub. No.: US 2013/0029389 A1
(43) Pub. Date: Jan. 31, 2013

(54) METHOD FOR PRODUCING D TYPE LACTIC ACID WITH HIGH OPTICAL PURITY

(71) Applicants: Chae Hwan Hong, Ansan (KR); Do Suck Han, Seongnam (KR); Si Hwan Kim, Anyang (KR)

(72) Inventors: Chae Hwan Hong, Ansan (KR); Do Suck Han, Seongnam (KR); Si Hwan Kim, Anyang (KR)

(73) Assignee: HYUNDAI MOTOR COMPANY, Seoul (KR)

(21) Appl. No.: 13/285,572

(22) Filed: Oct. 31, 2011

(30) Foreign Application Priority Data
Jul. 27, 2011 (KR) 10-2011-0074749

(51) Int. Cl. C12P 7/56

(52) U.S. Cl. 435/139

(57) ABSTRACT

The present invention provides a method for producing D-type lactic acid by using E. coli and glucose derived from a plant as active ingredient, in which D-type lactic acid is a raw material of D-type polylactic acid in order to produce a stereo-complex polylactic acid blend which is a valuable material as the plant-based material for the manufacture of interior and exterior parts of an automobile. More specifically, the present invention relates to a bacterial strain and a fermentation process for producing D-type lactic acid with high optical purity.

### CROSS-REFERENCE TO RELATED APPLICATION

This application claims the benefit of Korean Patent Application No. 10-2011-0074749, filed Jul. 27, 2011, under 35 U.S.C. §119(a). The entire contents of the aforementioned application are incorporated herein by reference.

### BACKGROUND

(a) Technical Field

The present invention relates to a method for producing D-type lactic acid via fermentation using E. coli.

(b) Background Art

There has been a sharp increase in the use of fossil fuel worldwide due to the rapid industrial development along with population growth, which has ensued a growing concern on how to deal with environmental pollution by an enormous amount of wastes and global warming caused by greenhouse-gas emissions. The U.S. Census Bureau has recently revealed that the world population has continued to grow showing about 2 billion people in 1800s, about 6.6 billion in 1999, about 6.7 billion people in 2008, and it is expected to reach about 7 billion in 2012.

Modern civilization has been closely associated with oil as an energy resource. According to the recent statistics, the world's oil reserve was about 1.2 trillion barrel as of the end of 2004 and its Reserve/Production may be depleted in about 40 years based on the current rate of production. Further, there has been an oil price hike, from about $17 US/barrel in 1995, about $60 US/barrel in 2006, and to about $100 US/barrel in 2008.

Since oil is not a renewable resource it will be eventually depleted in the future. Furthermore, the fuels and substances produced from fossil resources have been the main culprit increasing the global carbon dioxide emissions.

Plant-derived biomass polymers derived from renewable plant resources, such as corn, bean, sugar cane, wood, and the like, by a chemical or biological process, are known to be effective in helping to reduce global carbon dioxide emissions and are also environmentally use in their biodegradability. A polylactic acid or polyactide of biomass polymer is a linear aliphatic polyester that can be obtained by fermentation of starch in corn and potato. It may be obtained by polymerization of sugar monomers obtained via fermentation after saccharification from plant cellulose; and is a carbon-neutral eco-friendly thermoplastic polymer material.

The polylactic acid, first synthesized in 1940s, has been very limited in its uses, such as drug delivery system and a suture, due to its high production cost and rarity. A venture company, Cargill Dow Polymer (renamed as NatureWorks since December 2007), was established as a joint venture between the U.S. Cargill and Dow Chemical in 1997. The company completed installing facilities capable of producing 140,000 tons/year of polylactic acid in 2002. Since then it has been involved in mass-production of polylactic acid for various uses, such as films, cups, food containers, packaging, etc., along with continued effort in its research and development.

However, the physical properties of the polylactic acid are inferior to those of a general-purpose polymer material, resulting in limited applications. In particular, to be used as a material for automobiles, improvements of heat resistance and impact resistance would be required.

To resolve the above problems, a technique has been developed for blending optical isomers of L-type polylactic acid (PLLA) and D-type polylactic acid (PDLA). Polylactic acid production has been largely limited to PLLA, thus leaving D-type polylactic acid (PDLA) production very rare. This is because the low priced D-type lactic acid, a raw material for PDLA, cannot be produced in bulk. Therefore, there is an urgent need for the development of a fermentation technique for the production of low cost D-type lactide.

Lactic acid has been produced by either a biotechnological fermentation or a petrochemical synthesis. In petrochemical synthesis, the lactic acid is produced by a series of sequential reactions, i.e., ethylene derived from a crude oil is converted into acetaldehyde by oxidation, then into lactonitrile by hydrocyanation, purified and distilled and then finally hydrolyzed under hydrochloric acid or sulfuric acid to finally obtain lactic acid. However, the produced lactic acid is a racemic mixture consisting of 50% of D-type lactic acid and 50% of L-type lactic acid, which is a DL-type lactic acid without optical activity, and the above petrochemical synthesis is not capable of regulating the composition of the mixture. On the other hand, the biotechnological fermentation process can selectively produce lactic acid by using a specific microorganism in the presence of a carbohydrate substance obtained from nature, such as starch, a sugar cane, and the like as a substrate.

The conventional study has been mainly focused on L-type lactic acid fermentation. Recently, Toyota Motor Corporation has filed a patent application on the production of D-type lactic acid.

U.S. patent application Ser. No. 12/324,804 (U.S. Pat. No. 7,964,382) discloses a method for producing D-lactic acid involving gene manipulation of a gene containing D-lactate dehydrogenase. However, the patent does not provide detailed results on the physical properties of the products obtained by fermentation. Therefore, there is an urgent need for the development of a method for stable production of D-type lactic acid with high optical purity, under economically feasible conditions.

### SUMMARY OF THE DISCLOSURE

The present invention provides a method for producing D-type lactic acid, or a salt thereof, via fermentation by using E. coli. In particular, the present invention provides a method for producing D-type lactic acid with high optical purity.

Other objects and advantages according to the present invention will be described in more detail with reference to the following detailed description, claims and figures of the present invention.

In one aspect, the present invention provides a method for producing D-type lactic acid, or a salt thereof, with optical purity, comprising the step of fermenting and degrading a sugar component as a hydrocarbon source by culturing E. coli (KCTC 2223) under constant temperature conditions. In certain embodiments, the method includes constant pH conditions.

The features and advantages of the present invention may be summarized as follows:
(i) The present invention provides a method for the production of D-type lactic acid with high optical purity via fermentation using E. coli in the presence of a sugar component as a carbon source.
(ii) The method according to the present invention has an industrial advantage in terms of cost-effectiveness in producing D-type lactic acid with an improved productivity and high optical purity, which can serve as a raw material for the manufacture of D-type polylactic acid (PLA).

### BRIEF DESCRIPTION OF THE DRAWINGS

The above and other features of the present invention will now be described in detail with reference to certain exemplary embodiments thereof. The drawings are meant for illustration purposes only, and are not meant to limit the invention.

FIG. 1 is a schematic diagram of a process for producing D-type lactic acid with high optical purity via fermentation using E. coli according to an embodiment of the present invention; and

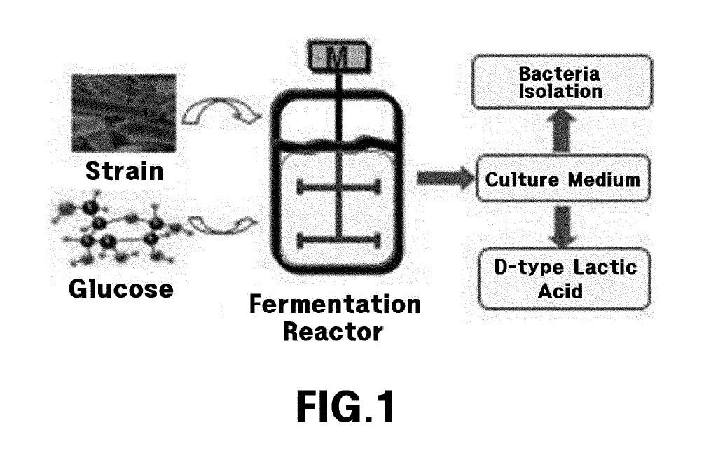

FIG. 2 is a graph showing an output of D-type polylactic acid according to each fermentation time obtained via fermentation using E. coli according to an embodiment of the present invention.

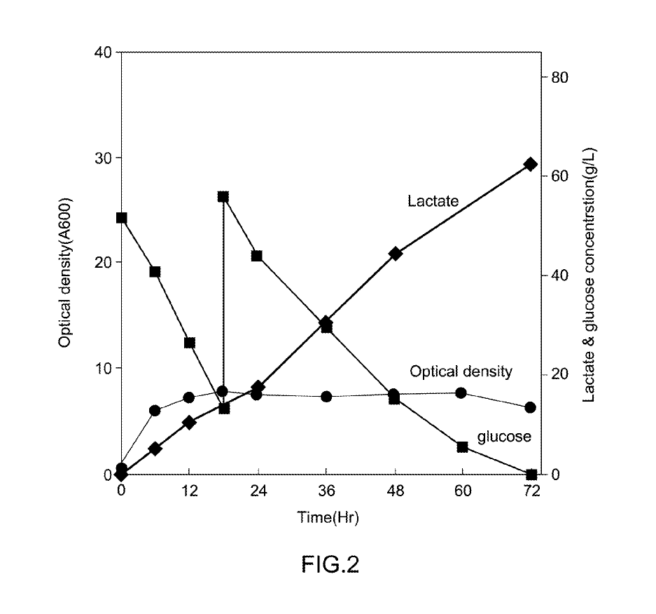

It should be understood that the appended drawings are not necessarily to scale, presenting a somewhat simplified representation of various preferred features illustrative of the basic principles of the invention. The specific design features of the present invention as disclosed herein, including, for example, specific dimensions, orientations, locations, and shapes will be determined in part by the particular intended application and use environment.

In the figures, reference numbers refer to the same or equivalent parts of the present invention throughout the several figures of the drawing.

### DETAILED DESCRIPTION

Hereinafter reference will now be made in detail to various embodiments of the present invention, examples of which are illustrated in the accompanying drawings and described below. While the invention will be described in conjunction with exemplary embodiments, it will be understood that present description is not intended to limit the invention to those exemplary embodiments. On the contrary, the invention is intended to cover not only the exemplary embodiments, but also various alternatives, modifications, equivalents and other embodiments, which may be included within the spirit and scope of the invention as defined by the appended claims.

In an exemplary embodiment, the present invention provides a method for producing D-type lactic acid, or salt thereof, with optical purity comprising the step of fermenting and degrading a sugar component as a hydrocarbon source by culturing E. coli (KCTC 2223) under constant temperature conditions. In certain embodiments, constant pH conditions are used.

The inventors of the present invention have discovered that D-type lactic acid with optical purity and high selectivity can be obtained as a fermentation product by using E. coli.

In certain embodiments, the fermentation is performed at 25° C.-100° C. In certain embodiments, the fermentation is performed at 25° C.-50° C. In certain embodiments, the fermentation is performed at 30° C.-45° C. In certain embodiments, the fermentation is performed at 35° C.-40° C. In various embodiments, the pH of the fermentation ranges from about 4.0-8.0. In various embodiments, the pH of the fermentation ranges from about 4.0-6.5. In various embodiments, the pH of the fermentation ranges from about 6.0-8.0. In various embodiments, the pH of the fermentation ranges from about 6.0-6.5. In still another exemplary embodiment, the fermentation is performed at 35° C.-37° C. under pH of 6.0-6.5.

In yet another exemplary embodiment, the pH can be adjusted by using ammonia water (NH₄OH).

The pH adjustment can be performed as follows; first, ammonia water is injected into the fermentation reactor through a pump operated according to the pH value being sensed in real time at a pH electrode installed inside the fermentation reactor by using a supply vessel that contains 10% aqueous ammonia concentration; and the ammonia water is injected inside the reactor until the supply pump is automatically stopped by sensing pH value of at least 6.0.

In still yet another exemplary embodiment, the sugars to be used include glucose, fructose, sucrose, mannose, or galactose, more preferably, glucose.

In a further exemplary embodiment, the D-type lactic acid with optical purity has 50% or higher of optical purity (50% enantiomeric excess (ee)). In a further exemplary embodiment, the D-type lactic acid with optical purity has 60% or higher of optical purity. In a further exemplary embodiment, the D-type lactic acid with optical purity has 70% or higher of optical purity. In a further exemplary embodiment, the D-type lactic acid with optical purity has 80% or higher of optical purity. In a further exemplary embodiment, the D-type lactic acid with optical purity has 90% or higher of optical purity. In a further exemplary embodiment, the D-type lactic acid with optical purity has 95% or higher of optical purity.

In a further exemplary embodiment, the D-type lactic acid with optical purity has 99% or higher of optical purity.

In another further exemplary embodiment, ammonia and salt forms in D-type lactic acid with optical purity are maintained after culturing and obtaining the D-type lactic acid with optical purity. As mentioned above, ammonia water as a pH adjuster reacts with lactic acid produced by injecting inside the reactor thereby forming a salt (NH₄⁺Lac⁻) by an acid-base bond.

In still another further exemplary embodiment, the culturing is performed in a batch process or a continuous process under anaerobic condition during the whole fermentation process.

The method for producing D-type lactic acid is as follows.

The fermentation culturing may be performed via a general batch process, a semi-batch process, or a continuous process at 30 to 50° C. and pH of 4.5 to 8.0 in an anaerobic condition; and when carbon sources are converted to lactic acid, the lactic acid can be isolated/concentrated from the culture solution through centrifugation, ultra filtration, decolorization, an ion exchange resin, electrodialysis, pervaporation, etc., to produce D-type lactic acid.

### DEFINITIONS

The term “chiral” refers to molecules which have the property of non-superimposability of the mirror image partner, while the term “achiral” refers to molecules which are superimposable on their mirror image partner.

The term “diastereomers” refers to stereoisomers with two or more centers of dissymmetry and whose molecules are not mirror images of one another.

The term “enantiomers” refers to two stereoisomers of a compound which are non-superimposable mirror images of one another. An equimolar mixture of two enantiomers is called a “racemic mixture” or a “racemate.”

The term “isomers” or “stereoisomers” refers to compounds which have identical chemical constitution, but differ with regard to the arrangement of the atoms or groups in space.

The terms “a,” “an,” and “the” refer to “one or more” when used in this application, including the claims. Thus, for example, reference to “a sample” includes a plurality of samples, unless the context clearly is to the contrary (e.g., a plurality of samples), and so forth.

Throughout this specification and the claims, the words “comprise,” “comprises,” and “comprising” are used in a non-exclusive sense, except where the context requires otherwise.

As used herein, the term “about,” when referring to a value is meant to encompass variations of, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Acids and bases useful in the methods herein are known in the art. Acid catalysts are any acidic chemical, which can be inorganic (e.g., hydrochloric, sulfuric, nitric acids, aluminum trichloride) or organic (e.g., camphorsulfonic acid, p-toluenesulfonic acid, acetic acid, ytterbium triflate) in nature. Acids are useful in either catalytic or stoichiometric amounts to facilitate chemical reactions. Bases are any basic chemical, which can be inorganic (e.g., sodium bicarbonate, potassium hydroxide) or organic (e.g., triethylamine, pyridine) in nature. Bases are useful in either catalytic or stoichiometric amounts to facilitate chemical reactions.

Preferred enantiomerically enriched compounds have an enantiomeric excess of 50% or more, more preferably the compound has an enantiomeric excess of 60%, 70%, 80%, 90%, 95%, 98%, or 99% or more. In preferred embodiments, only one enantiomer or diastereomer of a chiral compound of the invention is administered to cells or a subject.

### EXAMPLES

The following examples illustrate the invention and are not intended to limit the same.

### Example 1

The present invention uses a bacterial strain for a fermentation process for producing in a cost-effective way D-type lactic acid with high optical purity by using glucose as a carbon source.

In the present invention, E. coli (KCTC 2223) was used as a bacterial strain. The culture medium including tryptone (5.0 g), yeast extract (2.5 g), and sodium chloride (5.0 g), was prepared in a 1 L aqueous solution. The E. coli (KCTC 2223) was inoculated to the medium and cultured for about 5-6 hours to produce the starter (seed) culture medium at 100 g/L of glucose concentration. All the media and containers used for the fermentation were sterilized for 20 minutes at 120° C. before use. The culture medium used for the main fermentation reaction, comprising 50 g of glucose, 5 g of yeast extract, 0.5 g of magnesium sulfate, 5 g of ammonium chloride, and 0.1 g of an anti-foaming agent, was prepared in a 1 L aqueous solution. The culture medium of 5% (v/v) content was inoculated to the reactor for the main fermentation. The main fermentation reaction was performed at 37° C., and the pH of the culture was adjusted by using ammonia water. The fermentation reaction was performed under anaerobic condition during the entire process, and the pH of the culture was maintained at about 6.0-6.5. The fermentation was performed for 65-75 hours. The concentration of glucose component and D-type lactic acid in the fermentation medium were analyzed via High-Performance Liquid Chromatography (HPLC).

According to the above method of the present invention, about 60 g of D-type lactic acid was produced per 100 g of glucose for producing D-type lactic acid. The product obtained was 0.77 g/L·h and its purity was 99.1%.

The method of the present invention, by using glucose with high optical purity, enables to provide economical D-type lactic acid monomers, and thus can contribute to the development of a bio material which can prevent global carbon emissions.

### Comparative Example 1

D-type lactic acid was prepared by the main fermentation reaction under the same condition in Example 1 using the same E. coli strain except that oxygen was supplied for the initial 6 hours to prepare about 10% level of dissolved oxygen in the culture medium.

### Comparative Example 2

D-type lactic acid was prepared by the main fermentation reaction under the same condition in Example 1 using the same E. coli strain except that sodium hydroxide (NaOH) was used instead of ammonia water to adjust pH.

### Comparative Example 3

D-type lactic acid was prepared by the main fermentation reaction under the same condition in Example 1 using the same E. coli strain while maintaining the pH of the culture at 7.0.

The following Table 1 shows various concentration of D-type lactic acid according to different fermentation times in Example 1.

TABLE 1

| | D-type Lactic Acid |
|---|---|
| Time (hour) | Concentration (g/L) |
| 0 | 0 |
| 12 | 18 |
| 24 | 21 |
| 36 | 31 |
| 48 | 44 |
| 60 | 53 |
| 72 | 62 |

The following Table 2 shows various concentration (g/L) of D-type lactic acid after 72 hours of fermentation and optical purity according to Example 1, and Comparative Example 1 to Comparative Example 3.

TABLE 2

| | D-type | D-type Lactic |
|---|---|---|
| | Optical Purity | Acid Concentration |
| Section | (%) | After 72 hours (g/L) |
| Ex. 1 | 99 | 62 |
| Com. Ex. 1 | 98 | 40 |
| Com. Ex. 2 | 99 | 56 |
| Com. Ex. 3 | 98 | 53 |

The present invention has been described in detail with reference to exemplary embodiments thereof. However, it will be appreciated by those skilled in the art that changes may be made in these embodiments without departing from the principles and spirit of the invention, the scope of which is defined in the appended claims and their equivalents.

### Incorporation by Reference

The contents of all references (including literature references, issued patents, published patent applications, and co-pending patent applications) cited throughout this application are hereby expressly incorporated herein in their entireties by reference.

### EQUIVALENTS

Those skilled in the art will recognize, or be able to ascertain using no more than routine experimentation, many equivalents of the specific embodiments of the invention described herein. Such equivalents are intended with be encompassed by the following claims.

What is claimed is:

1. A method for producing D-type lactic acid, or salt thereof, with optical purity comprising the steps of fermenting and degrading a sugar component as a hydrocarbon source by culturing E. coli (KCTC 2223) under constant temperature conditions.

2. The method of claim 1, wherein the fermentation is performed at 35-37° C. and at a pH of 6.0-6.5.

3. The method of claim 1, further comprising a step of adjusting a pH using ammonia water (NH₄OH).

4. The method of claim 1, wherein the sugar component is glucose, fructose, mannose, galactose or sucrose.

5. The method of claim 1, wherein the D-type lactic acid with optical purity has 99% or higher of optical purity.

6. The method of claim 3, wherein the ammonia and salt forms in the D-type lactic acid with optical purity are maintained after culturing and obtaining the D-type lactic acid with optical purity.

7. The method of claim 1, wherein the culturing is performed in a batch process or a continuous process in anaerobic conditions during the whole fermentation production period.
